(19) United States
(12) Patent Application Publication
(10) Pub. No.: US 2016/0331484 A1
(43) Pub. Date: Nov. 17, 2016

(54) CANNULA AND METHOD FOR CONTROLLING DEPTH DURING SURGICAL PROCEDURES

(71) Applicant: Alan Ellman, Hewlett, NY (US)

(72) Inventor: Alan Ellman, Hewlett, NY (US)

(21) Appl. No.: 15/151,422

(22) Filed: May 10, 2016

Related U.S. Application Data
(60) Provisional application No. 62/161,800, filed on May 14, 2015.

(51) Int. Cl. A61B 90/00; A61M 25/00; A61M 25/09; A61B 18/14; A61M 25/06

(52) U.S. Cl. CPC A61B 90/03; A61B 18/1482; A61M 25/0662; A61M 25/09; A61M 25/0068; A61B 2018/00601

(57) ABSTRACT

A disc surgical system is provided having an elongated tubular member housing and an electrosurgical electrode for excising of or shrinking tissue. The instrument includes at least one cannula having an operative end for entering an operative field of a patient, at least one guide wire having a pointed end for piercing tissue, a tapered dilator configured to slide over a guide wire; and a trephine configured to slide over a guide wire. The operative end has threads on an exterior surface of the cannula for engagement with the tissue of the patient.

### CROSS REFERENCE TO RELATED APPLICATIONS

This application claims priority to U.S. Provisional Patent Application No. 62/161,800 filed on May 14, 2015, titled “CANNULA AND METHOD FOR CONTROLLING DEPTH DURING SURGICAL PROCEDURES”, the entirety of which is incorporated herein by reference.

### BACKGROUND

This invention relates to a cannula for a disc surgical system, and in particular, to a cannula for controlling depth of insertion into a surgical area. The present application incorporates U.S. Pat. No. 7,137,982 and 8,409,194 entirely by reference. The patented device as explained in the aforementioned patents is suitable for many spinal procedures. However, there is a need in the art for depth control and assistance to ensure proper depth precision into the spine during a surgical procedure.

### SUMMARY

A disc surgical system is provided having an elongated tubular member housing and an electrosurgical electrode for excising of or shrinking tissue. The instrument includes at least one cannula having an operative end for entering an operative field of a patient, at least one guide wire having a pointed end for piercing tissue, a tapered dilator configured to slide over a guide wire; and a trephine configured to slide over a guide wire. The operative end has threads on an exterior surface of the cannula for engagement with the tissue of the patient.

The various features of novelty which characterize the invention are pointed out with particularity in the claims annexed to and forming a part of this disclosure. For a better understanding of the invention, its operating advantages and specific objects attained by its use, reference should be had to the accompanying drawings and descriptive matter in which there are illustrated and described preferred embodiments of the invention, like reference numerals or letters signifying the same or similar components.

### BRIEF DESCRIPTION OF THE DRAWINGS

FIG. 1 is a plan view of the components of one form of surgical system of the invention, the system in this case being shown with an electrosurgical handpiece shown schematically connected to an electrosurgical generator;

FIG. 2 is a plan view of a dilator assembled to a cannula of the surgical system of FIG. 1;

FIG. 3 is a plan view of a dilator assembled to a different cannula of the surgical system of FIG. 1;

FIG. 4 is a plan view of a trephine assembled to a cannula of the surgical system of FIG. 1;

FIG. 5A is a perspective view of the tip of a cannula according to one aspect of the invention;

FIG. 5B is a perspective view of the tip of a cannula according to one aspect of the invention;

FIG. 5C is a perspective view of the tip of a cannula according to one aspect of the invention;

FIG. 5D is a perspective view of the tip of a cannula according to one aspect of the invention;

FIG. 5E is a perspective view of the tip of a cannula according to one aspect of the invention;

FIG. 6 is schematic views illustrating a step in a surgical procedure using the surgical system of FIG. 1;

FIG. 7 is schematic views illustrating a step in a surgical procedure using the surgical system of FIG. 1;

FIG. 8 is schematic views illustrating a step in a surgical procedure using the surgical system of FIG. 1;

FIG. 9 is schematic views illustrating a step in a surgical procedure using the surgical system of FIG. 1;

FIG. 10 is schematic views illustrating a step in a surgical procedure using the surgical system of FIG. 1;

FIG. 11 is schematic views illustrating a step in a surgical procedure using the surgical system of FIG. 1;

FIG. 12 is schematic views illustrating a step in a surgical procedure using the surgical system of FIG. 1;

FIG. 13 is schematic views illustrating a step in a surgical procedure using the surgical system of FIG. 1;

FIG. 14 is schematic views illustrating a step in a surgical procedure using the surgical system of FIG. 1;

FIG. 15 is schematic views illustrating a step in a surgical procedure using the surgical system of FIG. 1;

FIG. 16 is schematic views illustrating a step in a surgical procedure using the surgical system of FIG. 1; and

FIG. 17 is schematic views illustrating a step in a surgical procedure using the surgical system of FIG. 1.

### DETAILED DESCRIPTION OF PREFERRED EMBODIMENTS

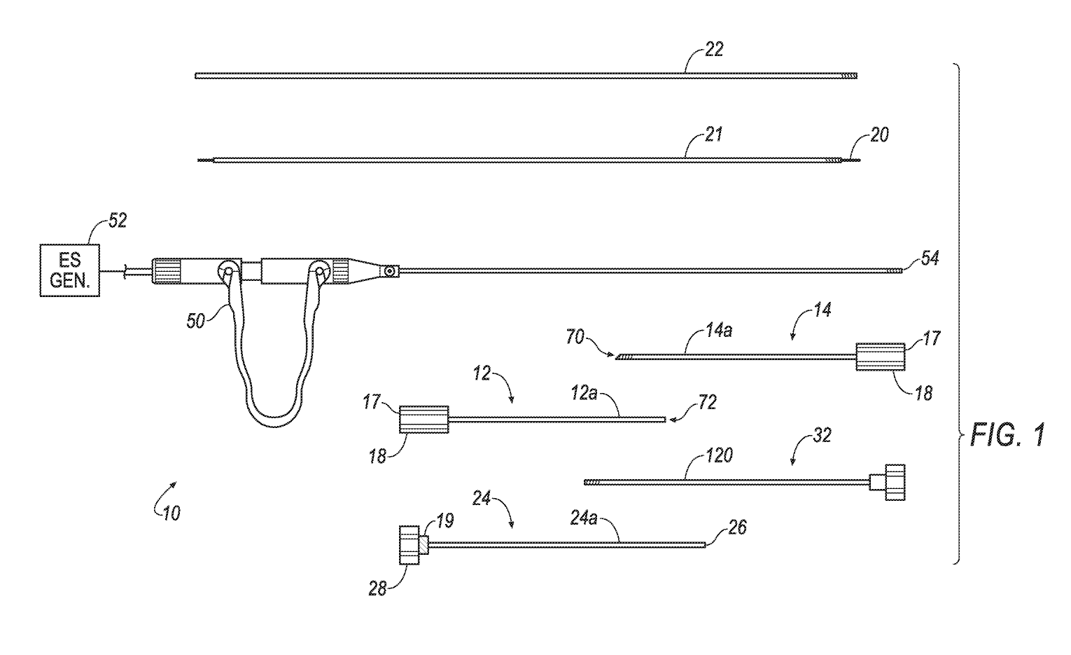

FIG. 1 illustrates the components for one form of a disc surgical system system 10 in accordance with one aspect of the invention. The system 10 includes electrosurgical handpiece 50, cannulas 12 and 14, dilator 24, guidewires 20 and 22, and trephine 32.

Cannula 12 has a straight end 72 and cannula 14 has a beveled end 70. Each of the cannulas 12 and 14 comprises an elongated straight tube 12a and 14a respectively that, in one example, has about 3.4 mm in outer diameter and a length of about 16.5 cm. The elongated straight tubes 12a and 14a are respectively connected to cannula heads 18. The end of the cannula heads 18 opposite the straight tubes 12a and 14a have an internally-threaded opening 17. A common bore or lumen, in one example, of about 3 mm in length extends through the straight tubes 12a and 14a and heads 18. Two guide wires are provided, one small guide wire 20 in a removable plastic tube 21 and one large guide wire 22. Each guide wire is solid with, in one example, an outer diameter of 1 and 1.3 mm respectively and about 40 cm long. Each guide wire 20 or 22 may have pointed ends for piercing tissue. It will be understood that the prior dimensions are by way of example only.

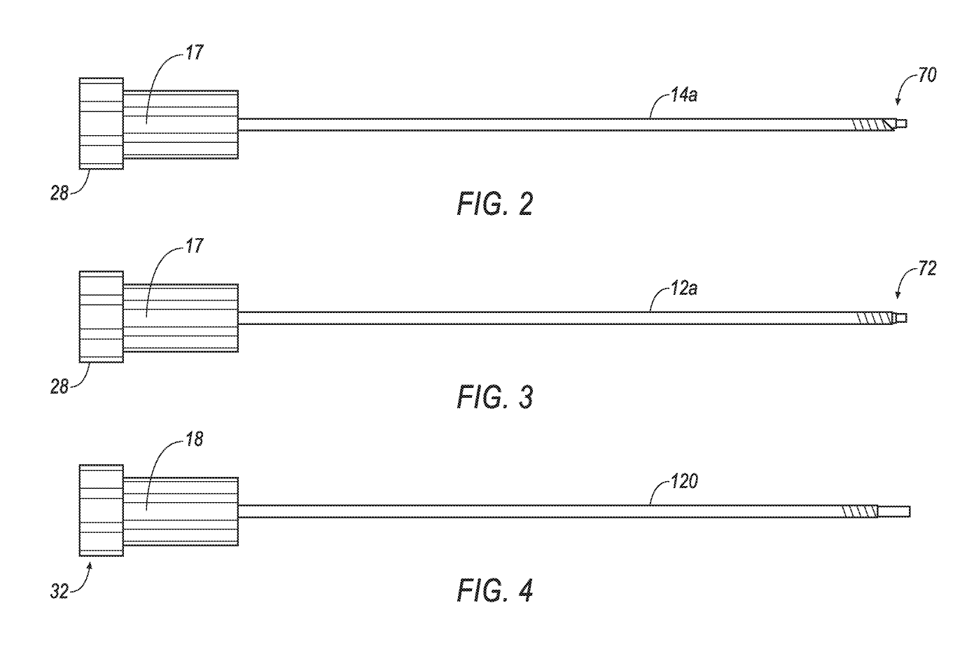

A dilator 24 includes a shaft 24a and is provided with a tapered tip 26 at one end of the shaft 24a and a dilator head 28 at an opposite end of the shaft 24a. The dilator 24 has a forwardly projecting threaded end 19 for removable connection to the internally-threaded opening 17 in the cannula head 18. When threaded together (as shown in FIGS. 2 and 3), a cannula 12 or 14 and the dilator 24 can be operated together as a single unit, or separated can operate as separate units.

As shown in FIG. 4, the system also comprises a trephine 32 having a straight tube 120 terminating in a beveled cutting edge. In one example, the dilator 12 or 14 has an OD of about 2.8 mm tapering down to about 2.2 mm. The trephine 32 has about the same OD. Their lengths are about 19.5 cm. The effort mentioned dimensions are by way of example only and should not be construed as limiting the invention.

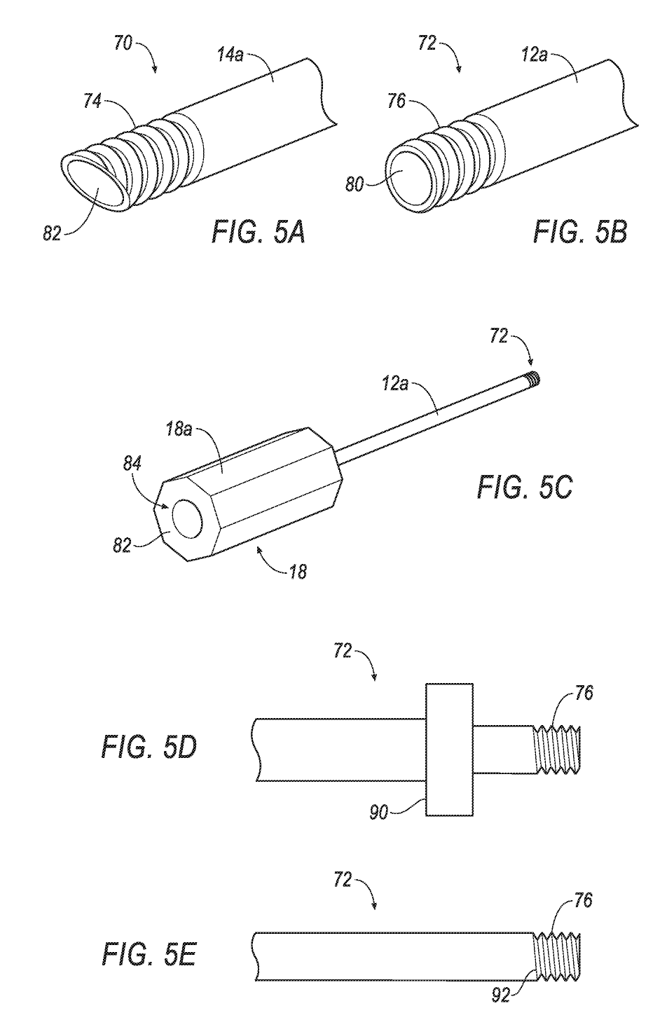

Referring now to FIG. 5A, one embodiment of a cannula 14 in accordance with the present invention is shown and described. In FIG. 5A, straight tube 14a is shown having a tip 70. In the depiction, tip 70 has a beveled configuration such that it is not perpendicular with respect to an axis of the straight tube 14a. Tip 70 has an aperture 82 that allows devices such as the dilator, trephine or other devices as described in the present invention to pass therethrough for surgical procedures. On an outer surface of the straight tube 14a proximate the tip 70, threads 74 are shown circumscribing the outer surface of the straight tube 14a. In one aspect, the threads have such a pitch to permit the cannula to advance a predefined distance per rotation of the cannula. For example, one rotation may translate into 1 mm of penetration. Likewise, different cannulas having different pitches of threads may be used for more or less depth penetration per rotation. Additionally, the threads may extend along the cannula a length that permits the cannula only to advance a certain depth. For example, as shown in FIG. 5d, the cannula may include a stop 90, which may be in the form of a simple bump, champfer, or other region that prevents the cannula from penetrating further than the stop. In FIG. 5e, the threads may extend up the surface a predefined length 92 (for example 3 mm) to ensure that no more than 3 of depth is achieved. The threads may also be clockwise or counterclockwise and may be of varying types, such as cutting threads.

With reference to FIG. 5B, straight tube 12a is shown having a tip 72 with a similar aperture 80 except that tip 72 is not beveled. Circumscribing the outer surface of the straight tube 14a proximate the tip 72 is threads 76.

Referring now to FIG. 5C, the tip as described in FIG. 5B is shown attached to cannula head 18. The cannula head 18 has an outer surface 18a engageable by a wrench or other device to permit rotation of the cannula such that threads 74 and 76, as will be described, engage the spinal area and permit the cannula to be screwed into position during surgical procedures.

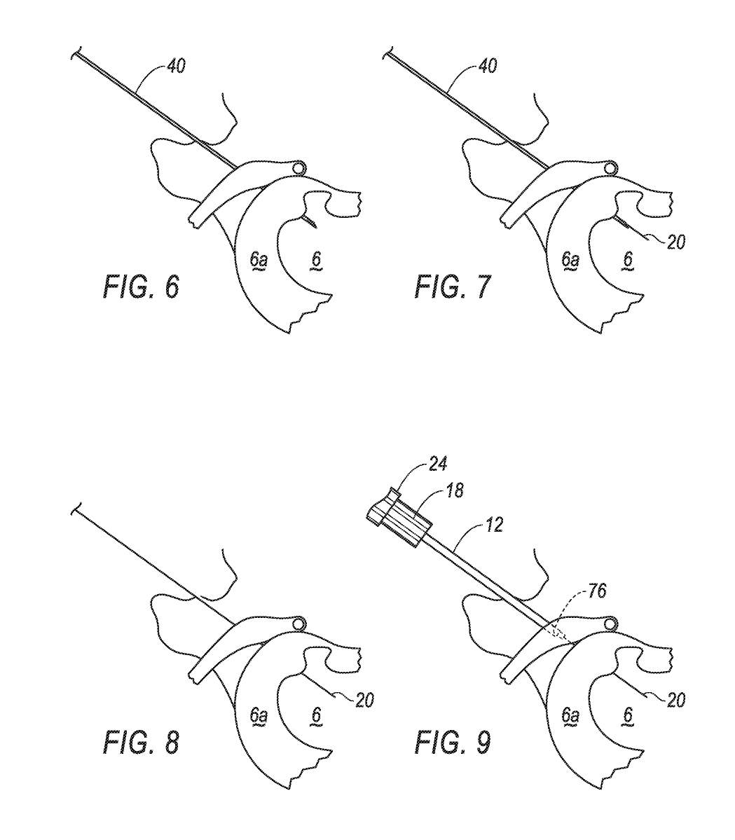

One procedure in accordance with the invention using these components is now described in connection with FIGS. 6-17 which show schematically a patient's back with a spinal disc comprising an annulus 6a surrounding the nucleus pulposus 6.

The patient may be positioned on a radiolucent table on a curved spinal frame in prone position or any other position suitable for the present procedure, the lumbar spine area prepped and draped in the usual sterile fashion, and the entry site marked, using, for example, a sterile marking pen 8-10 cm from midline on the affected side using fluoroscopic guidance. The skin, in one example, is then anesthetized with local anesthetic using a 25-gauge needle or other anesthetic. A spinal needle 40 is inserted through the marked entry point at a 45-degree angle to the skin (FIG. 6). The needle is advanced toward the foramen while the position is checked using both anterior/posterior (AP) and lateral fluoroscopy. The needle is then advanced into the disc using standard discography technique. The final position may be verified using fluoroscopy. In one example, discography is performed using 3 cc of contrast dye containing antibiotics and indigo carmine. A discogram may be performed to verify concordant pain and visualize disc morphology.

One of the guide wires 20 (FIG. 7) is threaded through the lumen of the needle 40 into the disc nucleus 6. Two different sizes of guide wires described previously can be provided (as well as additional guide wires) for use with spinal needles with different sized lumens. A skin incision is made at the needle site using, for example, a #11 scalpel. The needle 40 is subsequently removed leaving the guide wire 20 in place (FIG. 8). The cannula 12 (in the present example) and dilator 24 are joined together and are placed over the guide wire 20 and advanced toward the annulus 6a (FIG. 9). FIGS. 2 and 3 show a dilator 28 screwed and assembled to the head of cannulas 12 and 14. The respective lengths are such that the tapered end 26 of the dilator 24 protrudes, in one example, about 4 mm from the free end of the cannula 12 or 14.

For insertion of the cannula 12 or 14, the cannula is rotated such that the threads are driven into the spinal region. The depth of the cannula may be controlled based on the number of rotations or the depth markings to insert the cannula a desired distance into the operative area. It will be understood that the insertion of the cannula may be without the step of using the trephine 32 as the threads on the cannula will control depth.

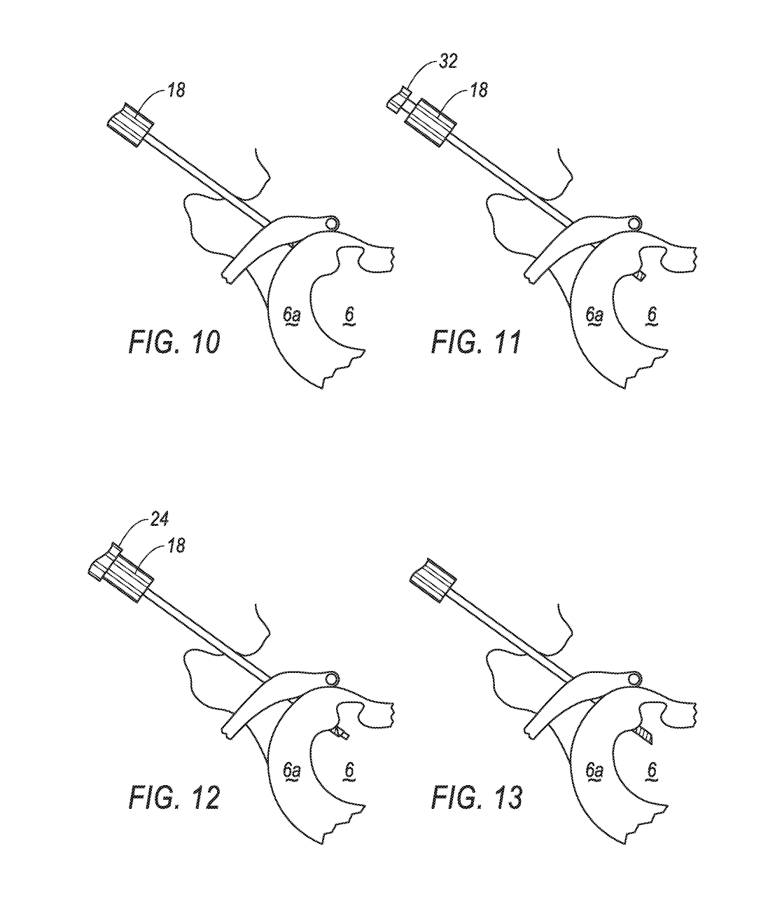

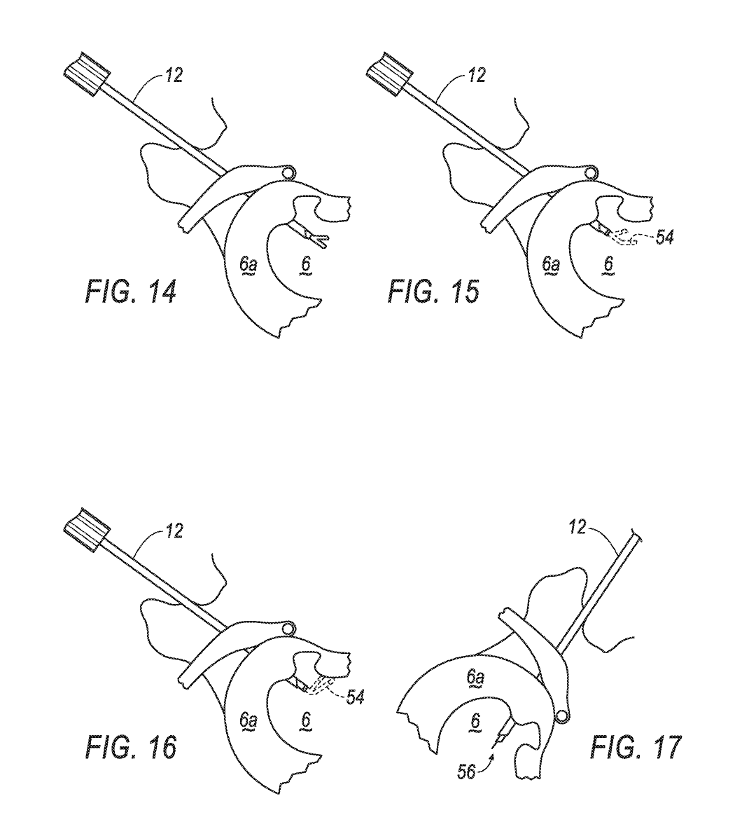

The dilator 24 (FIG. 10) is removed from the working cannula 12. The trephine 32 (FIG. 11) is inserted through the cannula 12 and advanced toward the outer surface of the disc annulus 6a. In one example, the trephine protrudes about 1 cm from the free end of the cannula. An annulotomy is created by applying slight pressure and a 360 degree rotation of the trephine, in one example, 1-3 turns. The trephine 32 is then removed and replaced by the dilator 24. The cannula 12 with dilator 24 is advanced under fluoroscopic guidance into the nucleus (FIG. 12) through rotation as specified above (if the trephine is used in conjunction with the cannula). When the dilator 24 is then removed, a portal into the disc is created (FIG. 13). A standard 2.5 mm diameter endoscopic grasping forceps (FIG. 14) can be used to manually extract nucleus material. A bipolar electrosurgical handpiece 50 as described in U.S. Pat. Nos. 6,231,571 and D562,978, the contents of which are herein incorporated by reference, an example of which is known commercially as the Trigger-Flex Bipolar System and available from Elliquence LLC, may be connected to an RF electrosurgical generator 52, also available commercially from Elliquence LLC., and set to an appropriate power setting (for example in the bipolar HEMO mode). The electrosurgical handpiece 50 may be included in the package with the other components or provided separately. The RF energy is activated while the handle is squeezed to extend and retract the electrode (FIG. 15), the active bendable electrode end 54 being deployed and retracted into the nucleus to create tracks of nucleus removal. In one example, the electrode tracks are directed into the 11:, 12:, 1:, 5:, 6: and 7: o′clock positions in order to accomplish nucleus pulposus decompression (although other configurations may be used). At a lower power setting, an annuloplasty can be performed at the annulus (FIG. 16). The electrosurgical handpiece is extracted from the cannula at the conclusion of the procedure. While stabilizing the skin around the cannula with the fingers of one hand, the other hand may slowly withdraw the cannula and dilator together if added. In one example, sutures are used to close the surgical site and a sterile bandage applied. The patient is provided with post-procedural instructions. FIG. 17 illustrates while the cannula is still in position that suction 56 can be provided to extract tissue.

The procedure may be performed under local anesthesia and/or conscious sedation to allow for patient monitoring for signs of nerve root irritation. Continuous fluoroscopic imaging in A/P and lateral views may be performed throughout the procedure to verify device positioning. Irrigation may be permitted to flow continuously during the procedure to ensure proper cooling of the disc space.

Either the small guide wire or the large guide wire may be inserted directly through the musculature toward the symptomatic disc. Once the guide wire is in the correct position within the disc, the chosen cannula and the tapered dilator, completely attached via the threaded proximal head, may be inserted. See FIG. 6 for component orientation.

Performing an annulotomy and to incise the annulus, the trephine 32 is placed over the guide wire and extended through the cannula 12. See FIG. 4. The trephine can be rotated with light pressure in a clockwise motion to incise the annulus. Once the incision is made, the trephine and guide wire are removed from the cannula and the cannula is advanced into the disc nucleus.

With the cannula confirmed in optimum position, the cannula is in place to perform a discectomy procedure.

In one aspect, the RF electrosurgical handpiece called Trigger-Flex System has on its shaft two etched markings (not shown) near the handle to aid in surgical depth monitoring:

Position 1: When the proximal (top) of the cannula head is flush to the distal etched marking, the cannula tip will be flush to the Trigger-Hex shaft.

Position 2: When the proximal (top) of the cannula head is flush to the proximal etched marking, the Trigger-Flex shaft will be exposed 1.0 cm beyond the cannula tip.

Position 3: When the proximal (top) of the cannula head is flush to the distal edge of the Trigger-Flex handle, the Trigger-Flex shaft will be exposed 3.3 cm beyond the cannula tip. The shaft has an overall length of about 23 cm and an OD of about 2.3 mm

It will be understood that the previously mentioned dimensions are by way of example only. To perform nucleoplasty, with the Trigger-Flex System in position at or in the nucleus, the handle is squeezed for full electrode advancement then retraction. This technique can be repeated for 5 passes in the disc while rotating the device. For annuloplasty; the Trigger-Flex System can be directed toward the inner annular wall in a sweeping motion.

While the Trigger-Flex System is described, other elongated electrosurgical handpieces can be substituted.

While the instrument of the invention is especially useful for spinal procedures, it is not limited to such uses and it will be understood that it can be employed in any electrosurgical procedure employing a cannula in MIS.

While the invention has been described in connection with preferred embodiments, it will be understood that modifications thereof within the principles outlined above will be evident to those skilled in the art and thus the invention is not limited to the preferred embodiments but is intended to encompass such modifications.

In this specification, various preferred embodiments may have been described with reference to the accompanying drawings. It will, however, be evident that various modifications and changes may be made thereto, and additional embodiments may be implemented, without departing from the broader scope of the invention as set forth in the claims that follow. The present invention is thus not to be interpreted as being limited to particular embodiments and the specification and drawings are to be regarded in an illustrative rather than restrictive sense.

It will be appreciated that the system and methods described herein have broad applications. The foregoing embodiments were chosen and described in order to illustrate principles of the methods and apparatuses as well as some practical applications. The preceding description enables others skilled in the art to utilize methods and apparatuses in various embodiments and with various modifications as are suited to the particular use contemplated. In accordance with the provisions of the patent statutes, the principles and modes of operation of this invention have been explained and illustrated in exemplary embodiments.

It is intended that the scope of the present methods and apparatuses be defined by the following claims. However, it must be understood that this invention may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future examples. Furthermore, all terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary. It is intended that the following claims define the scope of the invention and that the method and apparatus within the scope of these claims and their equivalents be covered thereby. In sum, it should be understood that the invention is capable of modification and variation and is limited only by the following claims.

What is claimed is:

1. An intervertebral disc surgical system for use with an electrosurgical instrument having an elongated tubular member housing and an electrosurgical electrode for excising of or shrinking tissue, comprising:
at least one cannula configured with a lumen to receive the elongated tubular member of the electrosurgical instrument, the cannula having an operative end for entering an operative field of a patient;
at least one guide wire having a pointed end for piercing tissue, wherein the guide wire is configured to fit within the lumen of the cannula;
a tapered dilator configured to slide over a guide wire; and
a trephine configured to slide over a guide wire,
wherein the operative end of the cannula has threads on an exterior surface of the cannula for engagement with the tissue of the patient.

2. The system according to claim 1, wherein the threads on the operative end extends a length along the cannula that corresponds to a depth to which the cannula is threaded into the tissue.

3. The system according to claim 1, wherein a distal end of the operative end has a face that is substantially perpendicular to an axis of the cannula.

4. The system according to claim 1, wherein a distal end of the operative end has a face that is not perpendicular to an axis of the cannula.

5. A spinal procedure comprising the steps:
providing a cannula having an operative end for entering an operative field of a patient, wherein the cannula has threads on an exterior surface of the cannula for engagement with tissue of a patient.
providing a guide wire, a tapered dilator with a lumen, a trephine with a lumen, and an elongated electrosurgical instrument;
threading the guide wire through a lumen in a needle into a disc nucleus after the needle is inserted into a patient's back toward a spinal disc;
removing the spinal needle leaving the guide wire in place;
joining the cannula and dilator together and placing the cannula and dilator over the guide wire;
advancing the cannula and dilator together toward the annulus by rotating the cannula and engaging the threads into the tissue of the patient;
removing the dilator to create a portal into the disc,
advancing the electrosurgical instrument through the cannula and into a pulpous to remove, shrink or modulate pulpous tissue.
